(19) United States
(12) Patent Application Publication
(10) Pub. No.: US 2004/0043433 A1
(43) Pub. Date: Mar. 4, 2004

(54) Light scatering determination of treatment potencies

(76) Inventor: Satish Deshpande, Ontario (CA)

Correspondence Address: DONALD F MOYER, 431 S DEARBORN 705, CHICAGO, IL, 60605

(21) Appl. No.: 10/416,099

(22) PCT Filed: Jan. 25, 2002
(86) PCT No.: PCT/US02/02132

(51) Int. Cl. G01N 33/53; G01N 33/567; G06K 9/00; C12M 1/34

(52) U.S. Cl. 435/7.2; 435/287.2; 382/128

(57) ABSTRACT

Potencies (44) of treatments effecting cells (11) are measured by difference (42) between angular intensity distributions of light scattered by treated cells and by untreated cells.

This application claims benefit of U.S. provisional application No. 60/260,761 filed Feb. 1, 2001.

The invention measures potencies of treatments which cause changes in cells by measuring difference between angular intensity distributions of light scattered by treated cells and by untreated cells.

This potency measurement is based on the unexpected discovery that treatments which cause the greatest difference between angular intensity distributions of light scattered by treated cells and by untreated cells have the greatest treatment potency.

This unexpected discovery is especially useful for determining which treatment will be most potent for a specific individual against the human immunodeficiency virus.

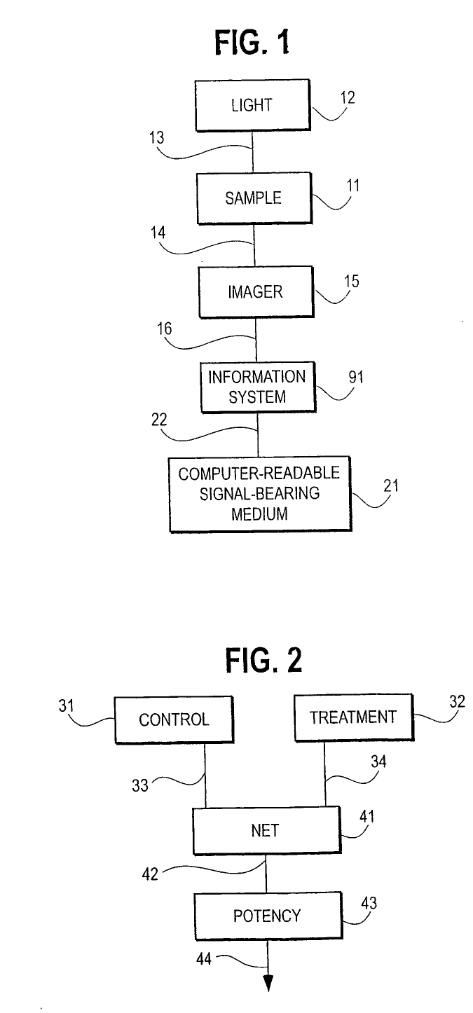

FIG. 1 schematically depicts a light scattering arrangement.

FIG. 2 schematically depicts a potency measurement.

Measuring potency of treatments on cells comprises illuminating cell samples, detecting light scattered by the samples, and causing output of a potency signal representing difference between light scattered by samples.

The cell samples comprise a control sample of cells, and a treatment sample of cells. The control sample is from a sample. The control sample can be the sample with nothing added. The control sample can be from the sample with a control treatment added. The control sample can be from the sample with a plurality of control treatments added. The treatment sample is from the sample with a treatment added. The treatment sample can be from the sample with a plurality of treatments added.

A “treatment” here—and throughout—means any substance which causes changes in parts of cells from the sample. The changes can be to surface parts of cells, to interior parts of cells, ant to combinations of these. The changes can be desired changes and can be undesired changes.

The control sample 11 is illuminated 13 with control light 12. A control imager 15 detects control scattered light 14. Control scattered light is a control portion of the control light which is scattered by the control sample and which is detected by the control imager.

The treatment sample 11 is illuminated 13 with treatment light 12. A treatment imager 15 detects treatment scattered light 14. Treatment scattered light is a treatment portion of the treatment light which is scattered by the treatment sample and which is detected by the treatment imager.

The control scattered light has a control angular intensity distribution 31. The treatment scattered light has a treatment angular intensity distribution 32. A potency signal 44 which represents difference between the control angular intensity distribution and the treatment angular intensity distribution is caused by the control imager and the treatment imager.

The potency signal can represent a difference—which can be positive and can be negative—between the control signal and the treatment signal at a single scattering angle. The potency signal can represent a plurality of differences—any of which can be positive and can be negative—between the control signal and the treatment signal at a plurality of scattering angles.

Experiments show that treatments which cause the greatest potency signals have the greatest treatment potency. For example, experiments show that—for a sample prepared from a specific HIV infected blood donor—the HIV treatment which causes the greatest potency signal has the greatest potency against HIV for that blood donor.

The control light and the treatment light can be from two separate light sources, can be from the one light source, and can be from portions of one light source. The control imager and the treatment imager can be two separate imagers, can be one imager, and can be portions of one imager. An imager—and imagers, and portions of am imager—can detect the control scattered light and the treatment scattered light concurrently using beam splitting means.

The control scattered light and the treatment scattered light must be comparable. This can be achieved in various ways. For example, the conditions of the two imagings can be interchangeable, except for the added treatment, so that the control scattered light and the treatment scattered light are directly comparable. For example, the two imagings can be calibrated with a standard imaging, so that the control scattered light and the treatment scattered light are comparable via the calibrations.

Various imaging means have been used. Scattered light can illuminate a screen which can be imaged. Scattered light can be imaged directly. Useful results are obtained using commercial video cameras for the imagings. Here imaging means need not form an image. The imager can be any means for detecting an angular intensity distribution of scattered light.

A control signal 33 can be caused by the control imager. The control signal represents the control angular intensity distribution of the control scattered light. A treatment signal can be caused by the treatment imager. The treatment signal 34 represents the treatment angular intensity distribution of the treatment scattered light.

The control signal can be subtracted electronically from the treatment signal to cause the potency signal. Difference between the treatment angular intensity distribution and the control angular intensity distribution can be imaged directly using beam splitting and phase shifting means.

The control imager and treatment imager can cause the potency signal via signal connection 16 with an information system 91 and via signal connection 22 of a computer-readable signal-bearing medium 21 with the information system.

The medium can cause use of the control signal and the treatment signal to cause output of the potency signal. The medium can have a net component 41. The net component can cause calculation of a net signal 42. The net signal represents difference between the treatment angular intensity distribution and the control angular intensity distribution. The medium can have a potency component 43. The potency component can cause calculation of the potency signal 44.

The potency signal represents difference between angular intensity distributions of light scattering by treated cells and by untreated cells. These difference can be represented in various ways. For example, the area under the curve of absolute values of positive and negative intensity differences at scattering angles can be calculated. For example, the curve of values of positive and negative intensity differences at scattering angles can be normalized to make the most negative intensity difference at least zero and the area under the normalized curve calculated. Various other analytical tools can be used.

Treatments which cause changes to interior parts of cells generate intensity difference at small angles. Treatments which cause changes to surface parts of cells generate difference at larger angles. In HIV treatments studied, effects on interior parts and on exterior parts are seen in scattering angles between zero and four degrees. Effects using other treatments and other cells can be seen at scattering angles of up to about 25 degrees. The range of scattering angles which should be studied depends on the cells and treatments being examined.

The potency signal can represent intensity difference between angular intensity distributions from various parings of control samples and treatment samples. The control samples can be the sample, can be from the sample with a control treatment added, and can be from the sample with a plurality of control treatments added. The treatment sample can be from the sample with a treatment added, and can be from the sample with a plurality of treatments added. The potency signal can represent difference between angular intensity distributions from various parings of these control and treatment sample variations.

The medium can have an historical component which causes calculation of an historical signal. The historical signal represents difference between historical data and a member from a signal group consisting of the treatment signal, the control signal,and the net signal. The historical signal can have components which can represent difference betweem historical data arind i mo n e o f the swal group, with each component from the components paired with a member from the signal group.

Historical data can comprise representations of historical control signals, representations of historical treatment signals, and representations of historical net signals. “Representations” can be various kinds of averages of historical data, constructions from historical data according to some criteria, bellwether data culled from historical data, and combinations of these.

Historical control, treatment, and net signals can be obtained by the means interchangeable with the means by which the control, treatment, and net signals are obtained. Historical control, treatment, and net signals can be obtained by the means comparable via calibrations with the means by which the control, treatment, and net signals are obtained.

Historical data can be used in the calculation of the potency signal. Historical data can be used in calculation of the net signal. Historical data can be used in causing the control signal and the treatment signal. Historical data can be used in configuring the imagings and in preparing the samples. In some cases historical data can be the control signal.

The historical component of the medium can be a component of the net component, can be a component of the potency component, can be a separate component of the medium, and can be combinations of these. The historical data can be part of the medium, can be available to the medium via a signal, and can be combinations of these.

A light scattering product for measuring effects of treatments on cells comprises the elements and configurations described above-which obtain the potency signal. The product operates according to a light scattering method for measuring effects of treatments on cells.

The light scattering method for measuring effects of treatments on cells comprises preparing a sample of cells, preparing a control sample of cells, preparing a treatment sample of Iki, tiluminating the control sample with control lt, illuminating the treatment sample with treatment light, detecting control scattered light with a control imager, detecting treatment scattered light with a treatment imager, and outputting a potency signal which represents difference between a treatment angular intensity distribution and a control angular intensity distribution by means equivalent to the means described above.

The light scattering method does not depend on how control and treatment samples are prepared, on how the angular scattering intensity distributions are obtained, nor on how difference between the distributions are determined. The light scattering product can use various means for preparing control and treatment sample and can use various elements and configurations to obtain the angular scattering intensity distributions and to determine difference between the distributions.

In one example, sample preparation starts with collection of about of five milliliters of blood held in an upright five milliliter tube with heparin at room temperature for about three to ten hours and then refrigerated at about four degrees Celsius until the red cells settle down and a Buffy coat is formed. The Buffy coat is removed with a Pasteur pipette and added to a tube with 10 millimoles per liter phosphate buffered saline to total typically 1.8 milliliters. This white cells solution is and kept on ice until measurements are taken.

Various other means for separating out the white cells can be used. The method and product can work without separating out the white cells.

About 1.2 milliliters of phosphate buffered saline is added to a 1.5 milliliter optical cuvette. This is assigned an optical density of zero percent. An amount of the white cells solution is added to increase the optical density by about 10 to 15 percent. This is the sample and is the control sample in this example.

The increase in optical density ensures that there are enough scattering cells to get useful scattering distributions, but not so many so that scattered photons which have been scattered by multiple cells dominate the scattered beam. The method and product can work with various cell densities and the resultant optical densities.shed by multie do cMethe beami Th methEd

The cuvette with the control sample is placed between the beam of a 780 nanometer laser and a video camera imager. About twenty images at fifteen second intervals are obtained. These images are averaged using the National Institutes of Health imaging analysis package ImageJ. This is the control signal which represents the control angular intensity distribution in this example.

Other wavelengths of laser light can be used. Other methods of imaging and averaging can be used. The method and product can work with any means of detecting the angular intensity distribution of the control scattered light.

A treatment—such as amprenavir—is added to the control sample in the cuvette so that the treatment concentration in the cuvette is 100 millimoles per liter. This is the treatment sample in this example.

Incubation times of fifteen minutes to twelve hours have been used. Thirty minutes works well for human HIV treatments. The incubation time depends on the cells and treatments.

The cuvette—now with the treatment sample—is placed between the beam of the 780 nanometer laser and the imager. About 20 images at 15 second intervals are obtained. These images are averaged using the National Institutes of Health imaging analysis package ImageJ. This is the treatment signal which represents the treatment angular intensity distribution in this example.

Other methods of imaging and averaging can be used. The method and product can work with any means for detecting and quantifying the angular intensity distribution of the treatment scattered light.

The control signal is subtracted from the treatment signal pixel by pixel. Along a radius from the center of the subtracted signal, averages along a chord centered on the radius are obtained. This is the net signal representing difference between the treatment angular intensity distribution and the control angular intensity distribution in this example.

The net signal can be obtained from the control signal and the treatment signal in various ways. The potency signal can be determined from the net signal by various means. The method and product can work with various determinations of difference between the control angular intensity distribution and the treatment angular intensity distribution.

A “signal” from a product part to a second product part—and a product part being “signal connected” with a second product part—here, and throughout, mean that a physical state of the product part causes a second physical state of the second product part. This can occur by various direct causal means and can occur by any of various transmission means. Transmitted signals can be any of various point-to-point and broadcast forms of energy transmission such as wireless and via wires, cables, and fibers. Parts of transmitted signals can reside with one form of the transmitted signal, parts can reside with a second form of transmitted signal, and parts can reside with various combinations of transmitted signals.

The several causes here can act via any of various processing modes. The processing can utilize configured processing elements such as fixed circuits, can utilize configurable processing elements such as field programmable gate arrays and neural networks, can utilize instructions in a data-bearing medium, and can utilize combinations of these. The processing can be by stand alone means, can act via a local information system, can act via a networked information system, and can act via combinations of these. The processing—in part at least—can be by parts of an imager. The computer-readable signal-bearing medium can be a transmitted signal, a data storage medium, and a combination of a transmitted signal and a data storage medium.

1. A light scatting product measuring effects of treatments on cells, the product comprising:
a sample of cells;
a control sample of cells,
the control sample being from the sample;
a treatment sample of cells,
the treatment sample being from the sample,
the treatment sample having a treatment added;
control light illuminating the control sample;
treatment light illuminating the treatment sample;
a control imager;
a treatment imager;
control scattered light,
the control scattered light being a control portion of the control light which is scattered by the control sample and which is detected by the control imager,
the control scattered light having a control angular intensity distribution;
treatment scattered light,
the treatment scattered light being a treatment portion of the treatment light which is scattered by the treatment sample and which is detected by the treatment imager,
the treatment scattered light having a treatment angular intensity distribution;
a potency signal,
the potency signal being caused by the control imager and the treatment imager imager,
the potency signal representing difference between the treatment angular intensity distribution and the control angular intensity distribution.

2. The product of claim 1 wherein:
the control imager adn the treatment imager cause the potency signal via a control signal, a treatment signal, and a computer-readable signal-bearing medium,
the control signal being caused by the control imager,
the control signal representing the control angular intensity distribution;
the treatment signal being caused by the treatment imager,
the treatment signal representing the treatment angular intensity distribution; and
the medium causes use of the treatment signal and the control signal to cause output of the potency signal.

3. The product of claim 2 wherein:
the medium has a net component,
the net component causing use of the control signal and the treatment signal to cause calculation of a net signal,
the net signal representing difference between the control angular intensity distribution and the treatment angular intensity distribution, and
the medium has an potency component,
the potency component causing calculation of the potency signal.

4. The product of claim 3 wherein the medium has an historical component which causes an historical signal, the historical signal representing difference between historical data and a member from a signal group consisting of the treatment signal, the control signal, and the net signal.

5. A light scattering product measuring effects of treatments on cells, the product comprising:
a sample of cells;
a control sample of cells,
the control sample being from the sample;
a treatment sample of cells,
the treatment samplebeing from the sample,
the treatment sample having a treatment added;
control light illuminating the control sample;
treatment light illuminating the treatment sample;
a control imager;
a treatment imager;
control scattered light,
the control scattered light being a control portion of the control light which is scattered by the control sample and which is detected by the control imager,
the control scattered light having a control angular intensity distribution;
treatment scattered light,
the treatment scattered light being a treatment portion of the treatment light which is scattered by the treatment sample and which is detected by the treatment imager,
the treatment scattered light having a treatment angular intensity distribution;
a potency signal,
the potency signal representing difference between the treatment angular intensity distribution and the control angular intensity distribution, the potency signal being caused by the control imager and the treatment imager via a control signal, a treatment signal, and a computer-readable signal-bearing medium,
the control signal being caused by the control imager,
the control signal representing the control angular intensity distribution;
the treatment signal being caused by the treatment imager,
the treatment signal representing the treatment angular intensity distribution;
a net component of the medium,
the net component causing use of the control signal and the treatment signal to cause calculation of a net signal,
the net signal representing difference between the control angular intensity distribution and the treatment angular intensity distribution, and
a potency component of the medium,
the potency component causing calculation of the potency signal.

6. The product of claim 5 wherein the medium has an historical component which causes an historical signal, the historical signal representing difference between historical data and a member chosen from a signal group consisting of the treatment signal, the control signal, and the net signal.

7. A light scattering method for measuring effects of treatments on cells, the method comprising:
preparing a sample of cells;
preparing a control sample of cells,
the control sample being from the sample;
preparing a treatment sample of cells,
the treatment sample being from the sample with a treatment added;
illuminating the control sample with control light;
illuminating the treatment sample with treatment light;
detecting control scattered light with a control imager,
control scattered light being a control portion of the control light which is scattered by the control sample and detected by the control imager,
the control scattered light having a control angular intensity distribution;
detecting treatment scattered light with a treatment imager,
treatment scattered light being a treatment portion of the treatment light which is scattered by the treatment sample and detected by the treatment imager,
the treatment scattered light having a treatment angular intensity distribution; and
outputting a potency signal,
the potency signal representing difference between the treatment angular intensity distribution and the control angular intensity distribution, and
the potency signal being caused by the control imager and the treatment imager.

8. the method of claim 7 wherein the step of outputting the potency signal comprises:
causing a control signal by the control imager,
the control signal representing the control angular intensity distribution;
causing a treatment signal by the treatment imager,
the treatment signal representing the treatment angular intensity distribution;
using the control signal and the treatment signal to cause output of the potency signal.

9. The method of claim 8 wherein the step of using the control signal and the treatment signal to cause output of the potency signal comprises:
calculating a net signal,
the net signal representing difference between the control signal and the treatment signal; and
using the net signal to cause output of the potency signal.

10. The method of claim 9 further comprising causing an historical signal, the historical signal representing difference between historical data and a member from a signal group consisting of the treatment signal, the control signal, and the net signal.

11. An imaging method for measuring effects of treatments on cells, the method comprising:
preparing a sample of cells;
preparing a control sample of cells,
the control sample being from the sample;
preparing a treatment sample of cells,
the treatment sample being from the sample with a treatment added;
illuminating the control sample with control light;
illuminating the treatment sample with treatment light;
detecting control scattered light with a control imager,
control scattered light being a control portion of the control light which is scattered by the control sample and detected by the control imager,
the control scattered light having a control angular intensity distribution;
detecting treatment scattered light with a treatment imager,
treatment scattered light being a treatment portion of the treatment light which is scattered by the treatment sample and detected by the treatment imager,
the treatment scattered light having a treatment angular intensity distribution;
outputting a potency signal,
the potency signal representing difference between the treatment angular intensity distribution and the control angular intensity distribution, and
the potency signal being caused by the control imager and the treatment imager by a method comprising:
causing a control signal by the control imager,
the control signal representing the control angular intensity distribution;
causing a treatment signal by the treatment imager,
the treatment signal representing the treatment angular intensity distribution;
calculating a net signal,
the net signal representing difference between the control signal and the treatment signal; and
using the net signal to cause output of the potency signal.

12. The product of claim 11 further comprising the step of causing an historical signal, the historical signal representing difference between historical data and a member from a signal group consisting of the treatment signal, the control signal, and the net signal.
